(19) United States
(12) Patent Application Publication
(10) Pub. No.: US 2024/0321403 A1
(43) Pub. Date: Sep. 26, 2024

(54) Method of Determining River Nitrous Oxide Emission based on Land-River-Atmosphere Simulation

(71) Applicant: Harbin Institute of Technology, Harbin City (CN)

(72) Inventors: Yu TIAN, Harbin (CN); Huihang SUN, Harbin (CN); Haoran ZHANG, Harbin (CN); Yiming MENG, Harbin (CN); Nanqi REN, Harbin (CN)

(21) Appl. No.: 18/605,776

(22) Filed: Mar. 14, 2024

(30) Foreign Application Priority Data
Mar. 14, 2023 (CN) 202310246459.9

(51) Int. Cl. G16C 20/20; G06Q 10/04

(52) U.S. Cl. CPC G16C 20/20; G06Q 10/04

(57) ABSTRACT

A method of determining nitrous oxide emission of a river based on land-river-atmosphere simulation, includes the steps of: obtaining nitrogen emission from land in each region; dividing the nitrogen emission into a prediction set and a test set; using nitrogen emission prediction set, and geographical variables and climate variables under the nitrogen emission prediction set to process RF regression model training to obtain a trained RF regression model, using nitrogen emission test set, and geographical variables and climate variables under the nitrogen emission test set to process RF regression model training to obtain a trained RF regression model, and outputting a river water quality concentration of each sub-basin in each region; obtaining river hydrological parameters of each sub-basin, inputting the river hydrological parameters and river water quality concentration of each sub-basin to an air-water interface gas exchange model to obtain a total river N₂O emission in each sub-basin.

### CROSS REFERENCE OF RELATED APPLICATION

This is a non-provisional application which claimed priority of Chinese application number 202310246459.9, filing date Mar. 14, 2023. The contents of these specifications, including any intervening amendments thereto, are incorporated herein by reference.

### BACKGROUND OF THE PRESENT INVENTION

### Field of Invention

The present invention relates to river N₂O emission calculation, and more particularly to an integrated technology of environmental engineering, environmental system simulation and prediction technology and computer technology for determining the river N₂O emission.

### Description of Related Arts

Nitrous oxide (N₂O) is a long-lived atmospheric trace gas that stays in the atmosphere for more than 100 years and has a strong greenhouse effect. The global warming potential of N₂O is 298 times that of CO₂, and it is currently the most important anthropogenic atmospheric pollutant causing stratospheric ozone depletion. River ecosystems have been identified as an important source of global N₂O. Current research showed that N₂O emissions from river ecosystems exceed 10% of total global anthropogenic N₂O emissions, but their formation and emission mechanisms are still unclear. Therefore, it is necessary to accurately identify and calculate river N₂O emissions and determine the spatial and temporal distribution patterns of river N₂O emissions in large-scale river basins, and provide precise pollution control strategies tailored to local conditions based on the findings of river N₂O emissions, which is very important for achieving water security.

The river N₂O emission factor (EF5) provided by the United Nations Intergovernmental Panel on Climate Change (IPCC) has been widely used in the past 20 years to estimate river N₂O emissions around the world. On the one hand, applying a constant emission factor can lead to overestimation or underestimation of N₂O emissions from the world's rivers, depending on when and where the emission factor is measured. On the other hand, the emission coefficient method ignores the function of rivers as links between land and atmospheric systems, cannot determine its nitrogen conversion efficiency based on the hydrological and water quality status of a specific river, and cannot achieve coordinated control of greenhouse gas pollution sources and pollution processes. Therefore, existing methods for estimating N₂O emissions from rivers in various places have poor accuracy. Using accurate and effective model tools to quantitatively describe the “land-river-atmosphere” nitrogen transfer process has become a key issue for accurate calculation and determination of river N₂O emissions.

### SUMMARY OF THE PRESENT INVENTION

An object of the present invention is to solve the problems of poor accuracy in measuring N₂O by using the existing methods, and to provide a method of determining river nitrous oxide emission based on land-river-atmosphere simulation.

A method of determining river nitrous oxide emission based on land-river-atmosphere simulation, the method comprises the steps of:
(1) obtaining nitrogen emission from land in each region;
(2) dividing the nitrogen emission from land into a nitrogen emission prediction set and a nitrogen emission test set; dividing geographical variables in each region into geographical variable prediction set and geographical variable test set; dividing climate variables in each region into climate variable prediction set and climate variable test set; training a RF regression model by using the nitrogen emission prediction set, geographical variable prediction set and climate variable prediction set to obtain a trained RF regression model, inputting the nitrogen emission test set, the geographical variable test set and the climate variable test set into the trained RF regression model, and outputting a river water quality concentration of each sub-basin in each region;
(3) obtaining river hydrological parameters of each sub-basin in each region, wherein the river hydrological parameters of each sub-basin comprises a water depth of the river, a flow velocity of the river, a water temperature of the river and a water surface area of the river; and
(4) providing an air-water interface gas exchange model and inputting the hydrological parameters of each sub-basin and water quality concentration of each sub-basin in each region of the river; and processing concentration conversion to obtain a totariver N₂O emission in each sub-basin in each region.

Preferably, the nitrogen emissions on land comprise urban residential anthropogenic nitrogen emission, industrial anthropogenic nitrogen emission, urban stormwater runoff non-point source nitrogen emission, rural residential nitrogen emission, crop farming nitrogen emission, and livestock farming nitrogen emission.

Preferably, the urban residential anthropogenic nitrogen emission is:

$$\{ \begin{matrix}{URN}_{discharge} = {URN}_{direct} + {URN}_{treat} \\ {URN}_{direct} = URPop \times {URCof}_{water} \times {URRate}_{direct} \times {URC}_{direct} \\ \begin{matrix}{URN}_{treat} = URPop \times {URCof}_{water} \times \\ {URRate}_{treat} \times ( 1 - {URRate}_{reuse} ) \times {URConc}_{treat}\end{matrix}\end{matrix} . \qquad Formula 1$$

In the formula, where URN_{discharge} refers to nitrogen discharge to water environment from urban residential area; URN_{direct} refers to a quantity of direct sewage discharge to the water environment without sewage treatment; URNₜᵣₑₐₜ refers to a quantity of nitrogen emission from urban residential area discharged to the water environment after sewage treatment by urban sewage treatment plant; URPop refers to an urban population; URCof_{water} refers to a domestic water coefficient of residents in urban area (per capita); URRate_{direct} refers to a ratio of direct sewage discharge to a total amount of sewage; URConc_{direct} refers to a nitrogen emission concentration of direct sewage discharge; URRateₜᵣₑₐₜ refers to a ratio of sewage treated by sewage treatment plants to a total sewage; URRateᵣₑᵤₛₑ refers to an effluent reuse rate of sewage treatment plant; URConcₜᵣₑₐₜ refers to a pollutant discharge concentration from the sewage treatment plant.

The urban stormwater runoff non-point source nitrogen emission is:

$$\{ \begin{matrix}{USRN}_{discharge} = {\sum }_{i = 1}^{4} {URSNRate}_{i} \times {UArea}_{i} \\ {USRNRate}_{i} = {NCon}_{i} \times {PDen}_{i , j} \times SF \times {AP}_{i} \times NCoef \\ {PDen}_{i , j} = 0.142 + 0.111 \times {DP}_{i}^{0.54} ( i = 1 ) \\ {PDen}_{i , j} = 1 ( i = 2 , 3 ) \\ {PDen}_{i , j} = 0.142 ( i = 4 ) \\ {UArea}_{i} = {UArea}_{2018} \times \frac{{UACoef}_{j}}{{UACoef}_{2018}}\end{matrix} . \qquad Formula 2$$

In the formula, wherein USRN_{discharge} refers to a nitrogen discharge from urban stormwater runoff surface source, USRNRateᵢ refers to a runoff nitrogen emission per unit area corresponding to a functional area i, where i=1, 2, 3, 4, which correspond to residential area, commercial area, industrial area and other area respectively; UArea; refers to a surface area of the functional area i; NConᵢ refers to a nitrogen emission concentration of the functional area i; PDen_{i, j} refers to an urban population density parameter of the functional area i in year j; SF refers to a cleaning frequency of urban community, where cleaning once a day is defined as 1; APᵢ refers to an annual precipitation (cm) in year j of the city where the functional area is located; NCoef refers to a nitrogen emission correction coefficient; PDent_{i, j} is a correction coefficient of the functional area; DPᵢ^{0.54} is a population density of an administrative region; UACoefⱼ refers to a correction coefficient of urban surface area in year j, UACoef₂₀₁₈ refers to a correction coefficient for urban surface area in 2018, UArea₂₀₁₈ refers to a surface area of the functional area in 2018.

The rural residential nitrogen emission is:

$$\{ \begin{matrix}{RRN}_{discharge} = {RRN}_{direct} + {RRN}_{treat} \\ {RRN}_{direct} = {RRN}_{total} \times {RRRate}_{direct} \\ {RRN}_{treat} = {RRN}_{total} \times {RRRate}_{treat} \times ( 1 - {RRCoef}_{removal} ) \\ {RRN}_{total} = RRPop \times ( {RRRate}_{DryT} \times {RRCoef}_{DryT} + \\ {RRRate}_{FlushT} \times {RRCoef}_{FlushT} )\end{matrix} . \qquad Formula 3$$

In the formula, RRN_{discharge} refers to a nitrogen discharge to the water environment from rural residential area; RRN_{direct} refers to a quantity of direct sewage discharge to the water environment without sewage treatment; RRNₜᵣₑₐₜ refers to a pollution discharge released to the water environment after treatment by rural sewage treatment facility; RRRateₜᵣₑₐₜ refers to a ratio of sewage treated by rural sewage treatment facility to a total sewage in rural area; RRCoefᵣₑₘₒᵥₐₗ refers to a pollutant removal rate of rural sewage treatment; RRPop refers to a total number of residents in rural area; RRRate_{DryT} refers to a ratio of dry toilets to total toilets in rural area; RRCoef_{DryT} refers to per capita pollutant emission coefficient of residents in rural area who use dry toilet; RRRate_{FlushT} refers to a ratio of flush toilets to total toilets in rural area; RRCoef_{FlushT} refers to a pollutant emission coefficient of residents in rural area who use flush toilet.

The crop farming nitrogen emission is:

$$\{ \begin{matrix}{CFN}_{discharge} = CFArea \times {CFCoef}_{discharge} \\ {CFCoef}_{discharge} = {CFCoef}_{discharge , 2017} \times \frac{{CFFertilizer}_{i}}{{CFFertilizer}_{2017}}\end{matrix} . \qquad Formula 4$$

In the formula, CFN_{discharge} refers to a pollution emission discharged into the water environment from crop farming; CFArea refers to a total sown area of farmland; CFCoef_{discharge} is a pollutant loss coefficient of farmland; CFCoef_{discharge, 2017} refers to a standard nitrogen loss coefficient of farmland based on data of the China's Second Pollution Source Census in 2017; CFFertilizerᵢ refers to a quantity of chemical fertilizer application in year i; CFFertilizer₂₀₁₇ refers to a quantity of chemical fertilizer application in 2017.

The livestock farming nitrogen emission is:

$$\{ \begin{matrix}{LFN}_{discharge} = {LFN}_{centralized} + {LFN}_{free} \\ {LFN}_{centralized} = {\sum }_{i = 1}^{6} {LFNumber}_{i} \times {LFRate}_{centralized , i} \times {LFCoef}_{centralized , i} \\ {LFN}_{free} = {\sum }_{i = 1}^{6} {LFNumber}_{i} \times {LFRate}_{free , i} \times {LFCoef}_{free , i}\end{matrix} . \qquad Formula 5$$

In the formula, LFN_{discharge} refers to a nitrogen discharge to the water environment from livestock farming; LFN_{centralized} is a pollution emission from centralized livestock farming; LFN_{free} is a pollution emission from free-range livestock farming; LFNumberᵢ is the number of fattening livestock i; LFRate_{centralized, i} is a ratio of centralized breeding quantity to total quantity of species i; LFCoef_{centralized, i} is a nitrogen emission coefficient of species i in centralized farming; LFRate_{free, i} is a ratio of free-range quantity to total quantity of species i; LFCoef_{free, i} is a nitrogen emission coefficient of species i in free-range livestock.

The industrial anthropogenic nitrogen emission is obtained by a specific process:
obtaining industrial nitrogen emissions in each region, and according to the proportion of the area of each sub-basin in each region to the region, converting the industrial nitrogen emissions in each region into the nitrogen emissions of each sub-basin, which is defined as the nitrogen discharged by industry into the water environment.

Preferably, in the step (2), the specific process of outputting a water quality concentration of each sub-basin in each region of the river is:
dividing the nitrogen emissions from the land in each region into nitrogen emission prediction set and nitrogen emission test set, dividing the environmental investment data in each region into an environmental investment data prediction set and an environmental investment data test set, and dividing the social statistical data on population and economy in each region into a prediction set of the social statistical data on population and economy, and a test set of the social statistical data on population and economy;
training the RF regression model by using the nitrogen emission prediction set, geographical variable prediction set, climate variable prediction set, environmental investment data prediction set, the prediction set of the social statistical data of population and economy to obtain the trained RF regression model; and then inputting the nitrogen emission test set, geographical variable test set, climate variable test set, environmental investment data test set, the test set of the social statistical data of population and economy to obtain the trained RF regression model and output the river water quality concentration of each sub-basin in each region.

Preferably, the environmental investment data comprises a proportion of environmental pollution investment and a number of environmental regulations;
the social statistical data on population and economy comprises a population density, a gross national product, a quantity of fertilizer application, a number of mobile phone households and a length of graded highway kilometers;
the geographic variables comprise a soil bulk density, soil organic matter, soil conductivity, soil pH, soil type proportion, land use proportion, maximum patch index, edge density, landscape shape index, Shannon diversity index and median landscape perimeter-to-area ratio;
the climate variables comprise an average temperature, an accumulated temperature greater than 10° C., an average rainfall, a humidity index and a normalized vegetation index.

Preferably, in step (3), the specific process of obtaining hydrological parameters of each sub-basin in each region of the river is:
inputting climate data of each sub-basin in each region into a SWAT model for hydrological parameter simulation, then outputting the river hydrological parameters of each sub-basin, wherein the climate data comprises rainfall temperature data, wind speed data, relative humidity data and solar radiation data.

Preferably, the total river N₂O emission in each sub-basin is:

$$\{ \begin{matrix}{F}_{{N}_{2} O} = {k}_{{N}_{2} O} \times ( {C}_{w} - {C}_{eq} ) \times 240 \\ {F}_{{N}_{2} Ototal} = {\sum }_{sub - basin} ( {\sum }_{month} {F}_{{N}_{2} O} \times SA \times N \times {10}^{- 15} ) \\ {C}_{w} = 0.42 \times {DIN}^{0.61} \\ {C}_{eq} = {C}_{air} \times {e}^{[ - 165.8806 + 222.8743 \times \frac{100}{{T}_{K}} + 92.0792 \times l n \frac{{T}_{K}}{100} - 1.48425 \times {( \frac{{T}_{K}}{100} )}^{2} ]} \times {M}_{{N}_{2} O} \\ {k}_{{N}_{2} O} = {k}_{600} \times {( \frac{{Sc}_{{N}_{2} O}}{600} )}^{- n} \\ {Sc}_{{N}_{2} O} = 2055.6 - 137.11 \times T + 4.3173 \times {T}^{2} - 0.05435 \times {T}^{3}\end{matrix} . \qquad Formula 6$$

In the formula, F_{N}_{₂}_{O} is a N₂O emission flux from the river to the atmosphere; C_{w} is the dissolved N₂O concentration in surface water of the river; C_{eq} is a theoretical N₂O concentration in surface water and atmospheric N₂O balance; k_{N}_{₂}_{O} is a N₂O gas transfer velocity; 240 is a unit conversion factor; F_{N}_{₂}_{Ototal} is a total N₂O emission in the basin in a given year (total N₂O emission in the basin refers to the sum of N₂O emission in all sub-basins, the basin is defined as the addition of all sub-basins); SA is a water surface area in a given sub-basin; N is a number of days in a given month; DIN is a dissolved inorganic nitrogen concentration simulated by the RF regression model; T_{K} is a water temperature simulated by SWAT; M_{N}_{₂}_{O} is a molecular weight of N₂O in N; Cₐᵢᵣ is a monthly scale N₂O concentration in air calculated based on a N₂O combined data set provided by NOAA Global Monitoring Laboratory; Sc_{N}_{₂}_{O} is a Schmidt number, T is a water temperature; n is an index; k₆₀₀ is a gas transfer velocity at 20° C.

Preferably, when n is ⅔, k₆₀₀=2.07+0.215×W₁₀^{1.7}, W₁₀ is a wind speed at 10m height.

Preferably, when n is ½, k₆₀₀=1.0+1.719×(V/H)^{0.5})+2.58×W₁₀, W₁₀ is a wind speed at 10 m height, V and H are flow velocity and water depth respectively.

The advantages of the present invention are as follows:
(1) The present invention provides a cross-media river N₂O emission estimation method, which breaks through the calculation boundary limitations of traditional methods. By using the influence of nitrogen discharged from land on nitrogen in rivers, the present invention accurately describes the land-river-atmosphere process of river N₂O emissions, achieves accurate simulation of river N₂O from source to emission process, and provides scientific means and technical tools for the coordinated control of land pollution, river pollution and air pollution.
(2) The RF regression model is used to obtain the river water quality concentration of each sub-basin, and the air-water interface gas exchange model is used to obtain the total river N₂O emissions of each sub-basin in each region. Therefore, the present invention provides a hybrid modeling method for river N₂O emissions based on machine learning and mechanism models, which fully utilizes the predictive performance of machine learning and mechanism models, effectively improves the calculation accuracy of river N₂O emissions calculation methods, and improves the calculation accuracy of river N₂O to monthly scale and catchment area resolution (<500 km²) in large-scale watersheds.
(3) The present invention combines statistical data with conventional meteorological monitoring data, and couples river hydrology and water quality processes with gas transfer laws, thereby avoiding the errors in calculation results caused by traditional methods that are deviated from actual laws, being capable of accurately identifying the greenhouse gas emission status of rivers in various regions, and providing reliable data support for pollution risk assessment, pollution traceability and pollution control strategies.
(4) The present invention considers the impact of atmospheric background N₂O concentration on river N₂O gas emissions, effectively reducing parameter uncertainty in the calculation process of river N₂O emissions. At the same time, the parameterized calculation formula and programmed calculation model make it possible to be applicable to various river basins in my country and can effectively promote the implementation of scientific pollution control work in my country.

### BRIEF DESCRIPTION OF THE DRAWINGS

FIG. 1 illustrates a flow chart of a method of determining river nitrous oxide emission based on land-river-atmosphere simulation according to a preferred embodiment of the present invention.

FIG. 2 illustrates a map of anthropogenic nitrogen emission at a sub-basin level in the Yangtze River Basin in 2019 as an image output on a display.

FIG. 3 is a graphical illustration of river DIN mean concentration map at a sub-basin level in the Yangtze River Basin in 2019 as an image output on a display.

FIG. 4 is a graphical illustration of a riverine dissolved N₂O concentration at a sub-basin level in the Yangtze River Basin in 2019 as an image output on a display.

FIG. 5 is a graphical illustration showing the simulation results of a river water surface area at a sub-basin level in the Yangtze River Basin in 2019 as an image output on a display.

FIG. 6 is a graphical illustration showing the calculation results of a gas transfer velocity at a sub-basin level in the Yangtze River Basin in 2019 as an image output on a display.

FIG. 7 is a graphical illustration showing the calculation results of a N₂O emission flux at a sub-basin level in the Yangtze River Basin in 2019 as an image output on a display.

FIG. 8 is a graphical illustration showing the calculation results of a N₂O emission at a sub-basin level in the Yangtze River Basin in 2019 as an image output on a display.

### DETAILED DESCRIPTION OF THE PREFERRED EMBODIMENT

The technical solutions in the embodiments of the present invention will be clearly and completely described below with reference to the accompanying drawings in the embodiments of the present invention. Obviously, the described embodiments are only some of the embodiments of the present invention, rather than all the embodiments. Based on the embodiments of the present invention, all other embodiments obtained by those of ordinary skill in the art without any creative work fall within the scope of protection of the present invention.

It should be noted that, as long as there is no conflict, the embodiments and features in the embodiments of the present invention can be combined with each other.

The present invention is further described in details below with the accompanying drawings and specific embodiments, but shall not be used as a limitation of the present invention.

### Embodiment 1

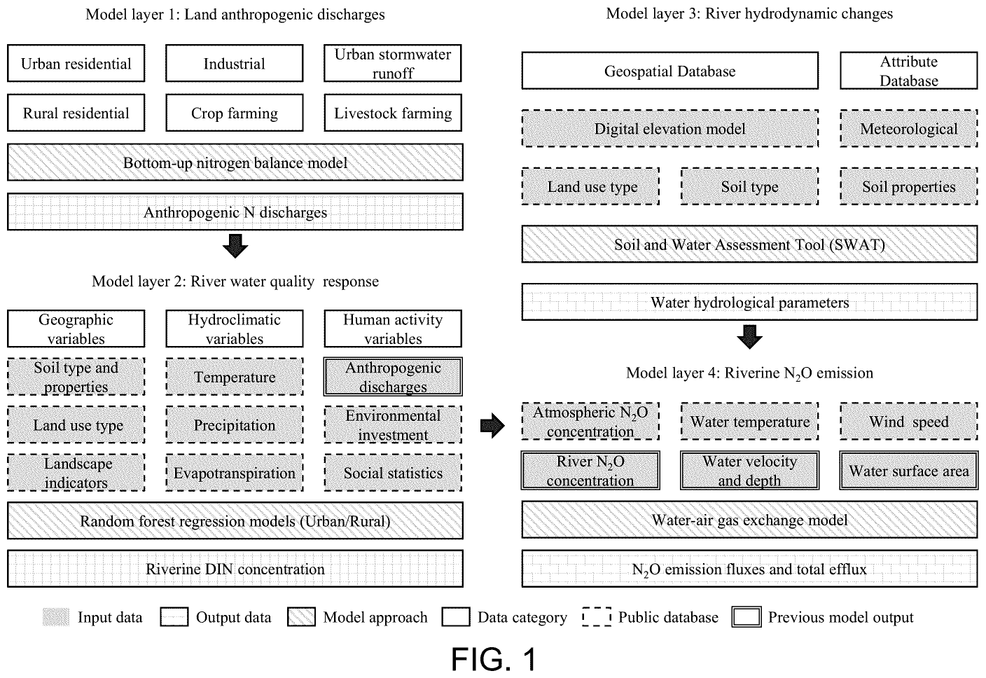

This embodiment is described with reference to FIG. 1. According to this embodiment of the present invention, a method of determining river nitrous oxide emission based on land-river-atmosphere simulation comprises the steps of:
(1) obtaining nitrogen emission from land in each region;
(2) dividing the nitrogen emission from land into a nitrogen emission prediction set and a nitrogen emission test set; dividing geographical variables in each region into geographical variable prediction set and geographical variable test set; dividing climate variables in each region into climate variable prediction set and climate variable test set; training a RF regression model by using the nitrogen emission prediction set, geographical variable prediction set and climate variable prediction set to obtain a trained RF regression model, inputting the nitrogen emission test set, the geographical variable test set and the climate variable test set into the trained RF regression model, and outputting a river water quality concentration of each sub-basin in each region;
(3) obtaining river hydrological parameters of each sub-basin in each region, wherein the river hydrological parameters of each sub-basin comprises a water depth of the river, a flow velocity of the river, a water temperature of the river and a water surface area of the river; and
(4) providing an air-water interface gas exchange model and inputting the hydrological parameters of each sub-basin and water quality concentration of each sub-basin in each region of the river; and processing concentration conversion to obtain a total river N₂O emission in each sub-basin in each region.

According to this embodiment, Random Forest (RF), as a mature machine learning algorithm, plays a key role in surface water quality prediction. Here, the present invention uses RF regression to predict water quality concentrations in surface water in each sub-basin of the watershed. Long-term monthly mean water quality concentrations are used as dependent variables for each sub-basin, and driver variables (geography, climate, and human activities) are used as independent variables. The training process and accuracy verification process of the model use a randomly and proportionally selected 80% and remaining 20% of the dataset. After model training and tuning, the predictive performance of each RF model is evaluated using multiple statistical metrics, including squared correlation coefficient (R²), root mean square error (RMSE), and mean absolute error (MAE). Specifically: input 80% of the data into the RF model, train the model, obtain the model parameters, and bring the model parameters into the model. When 20% of the data is input into the model, the square correlation coefficient (R²), root mean square error (RMSE) and mean absolute error (MAE) are used to compare the model outputs water quality concentration with the current concentration (the actual monitored water quality concentration value) to verify the accuracy of the resulting model.

According to FIG. 1, the input data is: digital elevation models in river hydrology, meteorological data, land use, soil type, soil properties, soil properties in river water quality response, temperature, anthropogenic emission, land use, rainfall, environmental input, landscape indicator, evaporation and social indicators, atmospheric N₂O concentration in river N₂O emission, hydrology, wind speed, river N₂O concentration, river flow speed, water depth and river surface area. The data in the dotted box is the data in the public database. Anthropogenic emissions, river N₂O concentration, river flow rate, water depth, and river water surface area represent the data output by the superior sub-model.

### Embodiment 2

The embodiment includes further limitations based on the Embodiment 1. According to this embodiment, nitrogen emissions on land comprises urban residential anthropogenic nitrogen emission, industrial anthropogenic nitrogen emission, urban stormwater runoff non-point source nitrogen emission, rural residential nitrogen emission, crop farming nitrogen emission, and livestock farming nitrogen emission.

According to this embodiment, the anthropogenic nitrogen emission model for terrestrial systems is a bottom-up model that quantifies the nitrogen input to rivers during human production and living processes, and takes into account the decentralized and centralized treatment processes of sewage. The sources of nitrogen emissions include six types of pollution sources, which are: urban residential, industrial, urban stormwater runoff surface sources, rural residential, crop farming and livestock farming. Anthropogenic nitrogen emission data based on county-level administrative boundaries are converted into sub-basin-level estimates using an urban-rural area weighting method. First, we calculate the county-level emissions of various pollution sources based on the county-level nitrogen emissions calculation framework. Second, the county-level administrative district boundary map and the sub-basin boundary map are combined, and the urban and rural areas of each intersecting unit are determined separately. Third, we disaggregate county-level anthropogenic emissions data into intersecting units based on identified urban and rural areas. Finally, we summarize anthropogenic nitrogen emission data for all target watersheds based on intersecting units located within the same watershed.

### Embodiment 3

The embodiment includes further limitations based on the Embodiment 2. According to this embodiment, the urban residential anthropogenic nitrogen emission is:

$$\{ \begin{matrix}{URN}_{discharge} = {URN}_{direct} + {URN}_{treat} \\ {URN}_{direct} = URPop \times {URCof}_{water} \times {URRate}_{direct} \times {URConc}_{direct} \\ {URN}_{treat} = URPop \times {URCof}_{water} \times {URRate}_{treat} \times ( 1 - {URRate}_{reuse} ) \times {URConc}_{treat}\end{matrix} … . \qquad Formula 1$$

In the formula, where URN_{discharge} refers to nitrogen discharged from urban life to the water environment; URN_{direct} refers to an amount directly discharged into the water environment without sewage treatment; URNₜᵣₑₐₜ refers to an amount of urban domestic nitrogen discharged into the water environment after treatment by the urban sewage treatment plant; URPop refers to an urban population; URCof_{water} refers to a domestic water coefficient of urban residents (per capita); URRate_{direct} refers to a ratio of direct sewage discharge to a total amount of sewage; URConc_{direct} refers to a nitrogen emission concentration of direct discharge sewage; refers to an ratio of sewage treated by sewage treatment plants to total sewage; URRateₜᵣₑₐₜ refers to a ratio of sewage treated by sewage treatment plants to a total sewage; URRateᵣₑᵤₛₑ refers to an effluent reuse rate; URConcₜᵣₑₐₜ refers to a concentration of pollutant discharged from the sewage treatment plant.

The urban stormwater runoff non-point source nitrogen emission is:

$$\{ \begin{matrix}{USRN}_{discharge} = {\sum }_{i = 1}^{4} {USRNRate}_{i} \times {UArea}_{i} \\ {USRNRate}_{i} = {NCon}_{i} \times {PDen}_{i , j} \times SF \times {AP}_{i} \times NCoef \\ {PDen}_{i , j} = 0.142 + 0.111 \times {DP}_{i}^{0.54} ( i = 1 ) \\ {PDen}_{i , j} = 1 ( i = 2 , 3 ) \\ {PDen}_{i , j} = 0.142 ( i = 4 ) \\ {UArea}_{i} = {UArea}_{2018} \times \frac{{UACoef}_{j}}{{UACoef}_{2018}}\end{matrix} . \qquad Formula 2$$

In the formula, USRN_{discharge} refers to a nitrogen discharge from urban stormwater runoff surface source, USRNRateᵢ refers to a runoff nitrogen emission per unit area corresponding to a functional area i, where i=1, 2, 3, 4, which correspond to living area, commercial area, industrial area and other area respectively; UArea; refers to a surface area of the functional area i; NConᵢ refers to a nitrogen emission concentration of the functional area i; PDen_{i, j} refers to an urban population density parameter of the functional area i in year j; SF refers to a cleaning frequency of urban community, where cleaning once a day is defined as 1; APᵢ refers to an annual precipitation (cm) in year j of the city where the functional area is located; NCoef refers to a nitrogen emission correction coefficient; PDen_{i, j} is a correction coefficient of the functional area; DPᵢ^{0.54} is a population density of an administrative region; UACoefⱼ refers to a correction coefficient of urban surface area in year j, UACoef₂₀₁₈ refers to a correction coefficient for urban surface area in 2018, UArea₂₀₁₈ refers to a surface area of the functional area in 2018.

The rural residential nitrogen emission is:

$$\{ \begin{matrix}{RRN}_{discharge} = {RRN}_{direct} + {RRN}_{treat} \\ {RRN}_{direct} = {RRN}_{total} \times {RRRate}_{direct} \\ {RRN}_{treat} = {RRN}_{total} \times {RRRate}_{treat} \times ( 1 - {RRCoef}_{removal} ) \\ {RRN}_{total} = RRPop \times ( {RRRate}_{DryT} \times {RRCoef}_{DryT} + {RRRate}_{FlushT} \times {RRCoef}_{FlushT} )\end{matrix} . \qquad Formula 3$$

In the formula, RRN_{discharge} refers to a nitrogen discharge to the water environment from rural residential area; RRN_{direct} refers to a quantity of direct sewage discharge to the water environment without sewage treatment; RRNₜᵣₑₐₜ refers to a pollution discharge released to the water environment after treatment by rural sewage treatment facility; RRRateₜᵣₑₐₜ refers to a ratio of sewage treated by rural sewage treatment facility to a total sewage in rural area; RRCoefᵣₑₘₒᵥₐₗ refers to a pollutant removal rate of rural sewage treatment; RRPop refers to a total number of residents in rural area; RRRate_{DryT} refers to a ratio of dry toilets to total toilets in rural area; RRCoef_{DryT} refers to per capita pollutant emission coefficient of residents in rural area who use dry toilet; RRRate_{FlushT} refers to a ratio of flush toilets to total toilets in rural area; RRCoef_{FlushT} refers to a pollutant emission coefficient of residents in rural area who use flush toilet.

The crop farming nitrogen emission is:

$$\{ \begin{matrix}{CFN}_{discharge} = CFArea \times {CFCoef}_{discharge} \\ {CFCoef}_{discharge} = {CFCoef}_{discharge , 2017} \times \frac{{CFFertilizer}_{i}}{{CFFertilizer}_{2017}}\end{matrix} … . \qquad Formula 4$$

In the formula, CFN_{discharge} refers to a pollution emission discharged into the water environment from crop farming; CFArea refers to a total sown area of farmland; CFCoef_{discharge} is a pollutant loss coefficient of farmland; CFCoef_{discharge, 2017} refers to a standard nitrogen loss coefficient of farmland based on data of the China's Second

Pollution Source Census in 2017; CFFertilizer, refers to a quantity of chemical fertilizer application in year i; CFFertilizer₂₀₁₇ refers to a quantity of chemical fertilizer application in 2017.

The livestock farming nitrogen emission is:

$$\{ \begin{matrix}{LFN}_{discharge} = {LFN}_{centralized} + {LFN}_{free} \\ {LFN}_{centralized} = {\sum }_{i = 1}^{6} {LFNumber}_{i} \times {LFRate}_{centralized , i} \times {LFCoef}_{centralized , i} \\ {LFN}_{free} = {\sum }_{i = 1}^{6} {LFNumber}_{i} \times {LFRate}_{free , i} \times {LFCoef}_{free , i}\end{matrix} . \qquad Formula 5$$

In the formula, LFN_{discharge} refers to a nitrogen discharge to the water environment from livestock farming; LFN_{centralized} is a pollution emission from centralized livestock farming; LFN_{free} is a pollution emission from free-range livestock farming; LFNumberᵢ is the number of fattening livestock i; LFRate_{centralized, i} is a ratio of centralized breeding quantity to total quantity of species i; LFCoef_{centralized, i} is a nitrogen emission coefficient of species i in centralized farming; LFRate_{free, i} is a ratio of free-range quantity to total quantity of species i; LFCoef_{free, i} is a nitrogen emission coefficient of species i in free-range livestock.

The industrial anthropogenic nitrogen emission is obtained by a process of:
obtaining industrial nitrogen emissions in each region, and according to the proportion of the area of each sub-basin in each region to the region, converting the industrial nitrogen emissions in each region into the nitrogen emissions of each sub-basin, which is defined as the nitrogen discharged by industry into the water environment.

According to this embodiment, for the urban residential anthropogenic nitrogen emission: through combining per capita domestic water consumption, urban sewage treatment rates, and urban sewage reuse rates, the nitrogen balance of urban domestic pollution emissions in any given year is determined. Also, the difference between uncollected sewage and collected sewage according to the sewage treatment rate is distinguished, and its calculation method is illustrated in Formula 1. The formula parameters in Formula 1 can be obtained from the “China Urban and Rural Construction Statistical Yearbook”, “China Environmental Statistical Yearbook”, statistical yearbooks of various provinces and the Second Pollution Source Census Manual.

For nitrogen emission from rural residential, the anthropogenic emission from rural residents is divided into direct emission and sewage treatment emission, as calculated in Formula 3.

For nitrogen emission from crop farming, the main source of anthropogenic nitrogen emission from farmland is rainfall erosion from chemical fertilizers, as calculated in Formula 4, wherein CFCoef_{discharge} is the pollutant loss coefficient of farmland per unit area; CFCoef_{discharge, 2017} is the standard nitrogen loss coefficient of farmland per unit area based on the data of the China's Second Pollution Source Census in 2017.

### Embodiment 4

The embodiment includes further limitations based on the Embodiment 2. According to this embodiment, in step (2), the specific process of outputting a river water quality concentration of each sub-basin in each region is:
dividing the nitrogen emissions from the land in each region into nitrogen emission prediction set and nitrogen emission test set; dividing the environmental investment data in each region into an environmental investment data prediction set and an environmental investment data test set; dividing the social statistical data on population and economy in each region into a prediction set of the social statistical data on population and economy, and a test set of the social statistical data on population and economy;
training the RF regression model by using the nitrogen emission prediction set, geographical variable prediction set, climate variable prediction set, environmental investment data prediction set, the prediction set of the social statistical data of population and economy to obtain the trained RF regression model; and then inputting the nitrogen emission test set, geographical variable test set, climate variable test set, environmental investment data test set, the test set of the social statistical data of population and economy to obtain the trained RF regression model and output the river water quality concentration of each sub-basin in each region.

### Embodiment 5

The embodiment includes further limitations based on the Embodiment 4. According to this embodiment, the environmental investment data comprises a proportion of environmental pollution investment and a number of environmental regulations;
the social statistical data on population and economy comprises a population density, a gross national product, a quantity of fertilizer application, a number of mobile phone households and a length of graded highway kilometers;
the geographic variables comprise a soil bulk density, soil organic matter, soil conductivity, soil pH, soil type proportion, land use proportion, maximum patch index, edge density, landscape shape index, Shannon diversity index and median landscape perimeter-to-area ratio;
the climate variables comprise an average temperature, an accumulated temperature greater than 10° C., an average rainfall, a humidity index and a normalized vegetation index.

According to this embodiment, the river water quality model includes 30 geographical, climate and human activity driver variables. These driver variables are monthly or annual time series. The selection of driver factors is based on a combination of its magnitude of impact on surface water DIN concentrations and data availability. The driver variables include geographical variables, including soil type and properties, land use type and landscape indicators, because they are widely recognized as potentially affecting river DIN concentration. Regarding climate variables, rainfall may directly affect the source and transport of pollutants by changing river flow, while temperature may indirectly affect water quality by affecting the transformation patterns and biochemical reaction rates of pollutants in water. Therefore, temperature, precipitation, and humidity index are considered as relevant climate variables. For human activity variables, in addition to relevant social statistics such as population and economy that are widely used in water quality predictions, we also consider anthropogenic nitrogen emissions and environmental investment data.

Environmental investment data also include the “Three Simultaneities” environmental protection investment. The “Three Simultaneities” system is a basic system for environmental management of construction projects and it is an important manifestation of the prevention-oriented environmental protection policy in my country (China), that is, environmental protection facilities in construction projects must be designed, constructed, and put into use simultaneously with the main project. The scope of application of the “Three Simultaneities” system includes: new, renovation, and expansion projects; technological transformation projects; and engineering projects that may cause pollution and damage to the environment.

### Embodiment 6

The embodiment includes further limitations based on the Embodiment 1. According to this embodiment, in step (3), the specific process of obtaining hydrological parameters of each sub-basin in each region of the river is:
inputting climate data of each sub-basin in each region into a SWAT model for hydrological parameter simulation, then outputting the river hydrological parameters of each sub-basin, wherein the climate data comprises rainfall temperature data, wind speed data, relative humidity data and solar radiation data.

According to this embodiment, the water surface area of the river in the river hydrological parameters of each sub-basin is calculated based on the river length and average width of each sub-basin.

SWAT (Soil and Water Assessment Tool) was developed in 1994 by Dr. Jeff Arnold of the Agricultural Research Center of the United States Department of Agriculture (USDA). The model was originally developed to predict the long-term impacts of land management on water, sediment, and chemicals in a large watershed with complex and variable soil types, land uses, and management practices. The SWAT model uses daily time for continuous calculation, and it is a distributed watershed hydrological model based on GIS. It has been rapidly developed and applied in recent years, mainly using the spatial information provided by remote sensing and geographic information systems to simulate a variety of different hydrological, physical and chemical processes, such as water quantity, water quality, and the transport and transformation process of pesticides.

### Embodiment 7

The embodiment includes further limitations based on the Embodiment 1. According to this embodiment, the total river N2O emission in each sub-basin refers to:

$$\{ \begin{matrix}{F}_{{N}_{2} O} = {k}_{{N}_{2} O} \times ( {C}_{w} - {C}_{eq} ) \times 240 \\ {F}_{{N}_{2} Ototal} = {\sum }_{sub - basin} ( {\sum }_{month} {F}_{{N}_{2} O} \times SA \times N \times {10}^{- 15} ) \\ {C}_{w} = 0.42 \times {DIN}^{0.61} \\ {C}_{eq} = {C}_{air} \times {e}^{[ - 165.8806 + 222.8743 \times \frac{100}{{T}_{K}} + 92.0792 \times l n \frac{{T}_{K}}{100} - 1.48425 \times {( \frac{{T}_{K}}{100} )}^{2} ]} \times {M}_{{N}_{2} O} \\ {k}_{{N}_{2} O} = {k}_{600} \times {( \frac{{Sc}_{{N}_{2} O}}{600} )}^{- n} \\ {Sc}_{{N}_{2} O} = 2055.6 - 137.11 \times T + 4.3173 \times {T}^{2} - 0.05435 \times {T}^{3}\end{matrix} . \qquad Formula 6$$

In the formula, F_{N}_{₂}_{O} is a N₂O emission flux from the river to the atmosphere; Cis the dissolved N₂O concentration in surface water of the river; C_{eq} is a theoretical N₂O concentration in surface water and atmospheric N₂O balance; k_{N}_{₂}_{O} is a N₂O gas transfer velocity; 240 is a unit conversion factor; IN, Ototal is a total N₂O emission in the basin in a given year (total N₂O emission in the basin refers to the sum of N₂O emission in all sub-basins, the basin is defined as the addition of all sub-basins); SA is a water surface area in a given sub-basin; N is a number of days in a given month; DIN is a dissolved inorganic nitrogen concentration simulated by the RF regression model; T_{K} is a water temperature simulated by SWAT; M_{N}_{₂}_{O} is a molecular weight of N₂O in N; Cₐᵢᵣ is a monthly scale N₂O concentration in air calculated based on a N₂O combined data set provided by NOAA Global Monitoring Laboratory; Sc_{N}_{₂}_{O} is a Schmidt number, T is a water temperature; n is an index; k₆₀₀ is a gas transfer velocity at 20° C.

### Embodiment 8

The embodiment includes further limitations based on the Embodiment 7. According to this embodiment, under low wind speed condition, n is ⅔. At this time, k₆₀₀=2.07×0.215×W₁₀^{1.7}, where W₁₀ is a wind speed at 10 m height.

### Embodiment 9

The embodiment includes further limitations based on the Embodiment 7. According to this embodiment, under high wind speed condition, n is ½, k₆₀₀=1.0+1.719×(V/H)^{0.5})+2.58×W₁₀, where W₁₀ is a wind speed at 10m height, V and H are flow velocity and water depth respectively.

Experimental verification:

The present invention has been successfully applied in the calculation and determination of N20 emissions from rivers in the Yangtze River Basin. The Yangtze River Basin is the largest river basin in Eurasia, with a population of more than 400 million and accounting for 42% of China's gross domestic product (GDP). The river area of the Yangtze River Basin exceeds 40,000 km², accounting for ¼ of China's total water area and 35% of China's surface water greenhouse gas emissions. Taking 2019 as an example, the anthropogenic nitrogen emissions, river water quality response, and river N₂O in the Yangtze River Basin are calculated.

The specific process are as follows:

### 1. Calculation of Terrestrial (Land) Anthropogenic Nitrogen Emissions

Using a bottom-up calculation model of anthropogenic nitrogen emissions, county-level terrestrial anthropogenic nitrogen emissions are calculated based on county-level basic statistical data in the Yangtze River Basin in 2019, wherein six pollution sources from urban residential, industries, urban stormwater runoff surface sources, rural residential, crop farming, and livestock farming are included. The calculation results of anthropogenic nitrogen emissions from various pollution sources in the Yangtze River Basin are shown in Table 1.

TABLE 1
Calculation results of anthropogenic nitrogen emissions from various
pollution sources in the Yangtze River Basin based on county-level
basic statistics in the Yangtze River Basin in 2019:

| | | | Storm- | | | Live- |
|---|---|---|---|---|---|---|
| | Urban | Indus- | water | Rural | Crop | stock |
| | residential | tries | Runoff | residential | farming | farming |
| Nitrogen | 28.8 | 4.2 | 3.66 | 17.1 | 27.3 | 20.0 |
| Emission | | | | | | |
| (10,000 | | | | | | |
| tons) | | | | | | |

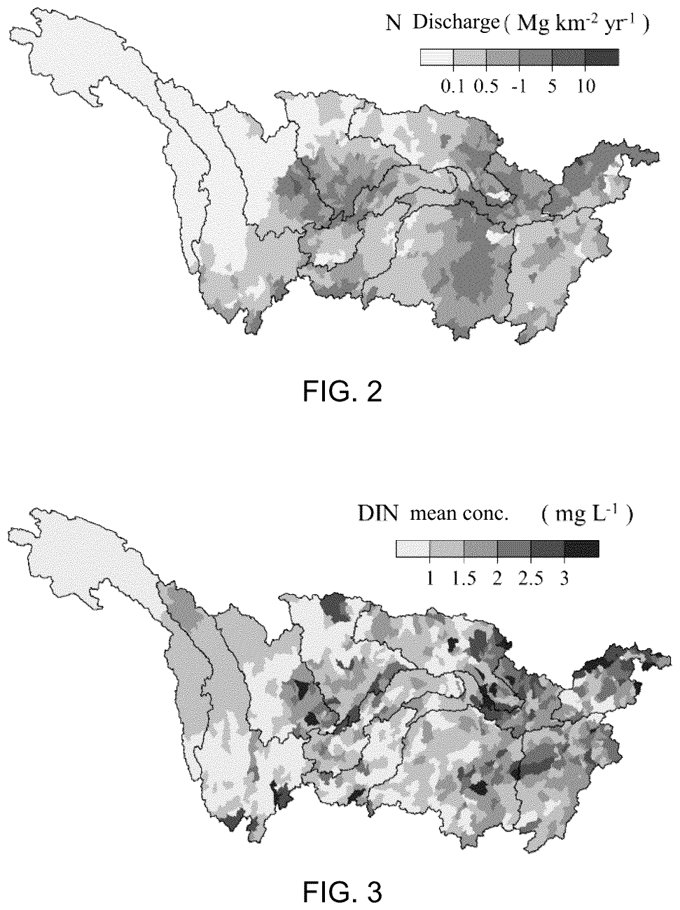

The anthropogenic nitrogen emission data based on county-level administrative boundaries are converted into sub-basin-level estimates using an urban-rural area weighting method. Based on the emissions of various pollution sources at the county level, combined with the county-level administrative district boundary map and the boundary map of the sub-basin, the urban and rural areas of each intersecting unit are determined respectively. Then, the County-level anthropogenic emissions data are disaggregated into intersecting units based on identified urban and rural areas. Finally, we summarized anthropogenic nitrogen emission data in the Yangtze River Basin based on the intersecting units located within the same basin. The anthropogenic nitrogen emissions at the sub-basin level are shown in FIG. 2.

According to the time emission pattern of pollution sources, the anthropogenic nitrogen emissions from various pollution sources are divided into monthly scales. The calculation results are shown in Table 2.

TABLE 2
Monthly calculation results of anthropogenic nitrogen emissions in the Yangtze River Basin:

| Month | January | February | March | April | May | June | July | August | September | October | November | December |
|---|---|---|---|---|---|---|---|---|---|---|---|---|
| Nitrogen | 5.8 | 5.9 | 6.3 | 9.4 | 9.6 | 9.4 | 7.6 | 7.3 | 6.6 | 6.4 | 6.0 | 5.9 |
| Emission | | | | | | | | | | | | |
| (10,000 | | | | | | | | | | | | |
| tons) | | | | | | | | | | | | |

### 2. Simulation of River Water Quality Response Process

Select 30 driver variables which includes 11 geographical variables, 5 climate variables and 15 human activity variables. The 11 geographical variables includes: soil bulk density, soil organic matter, soil conductivity, soil pH, soil type proportion, land use proportion, maximum patch index, edge density, landscape shape index, Shannon diversity index, median landscape perimeter-to-area ratio. The 5 climate variables includes: an average temperature, an accumulated temperature greater than 10° C., an average rainfall, a humidity index and a normalized vegetation index. The 15 human activity variables includes: urban residential nitrogen emission, industries nitrogen emission, urban stormwater runoff non-point source nitrogen emission, rural residential nitrogen emission, crop farming nitrogen emission, livestock farming nitrogen emission, a population density, a gross national product, a quantity of fertilizer application, a number of mobile phone households, a length of graded highway kilometers, a proportion of environmental pollution investment, a number of environmental regulations, and “Three Simultaneities” environmental protection investment.

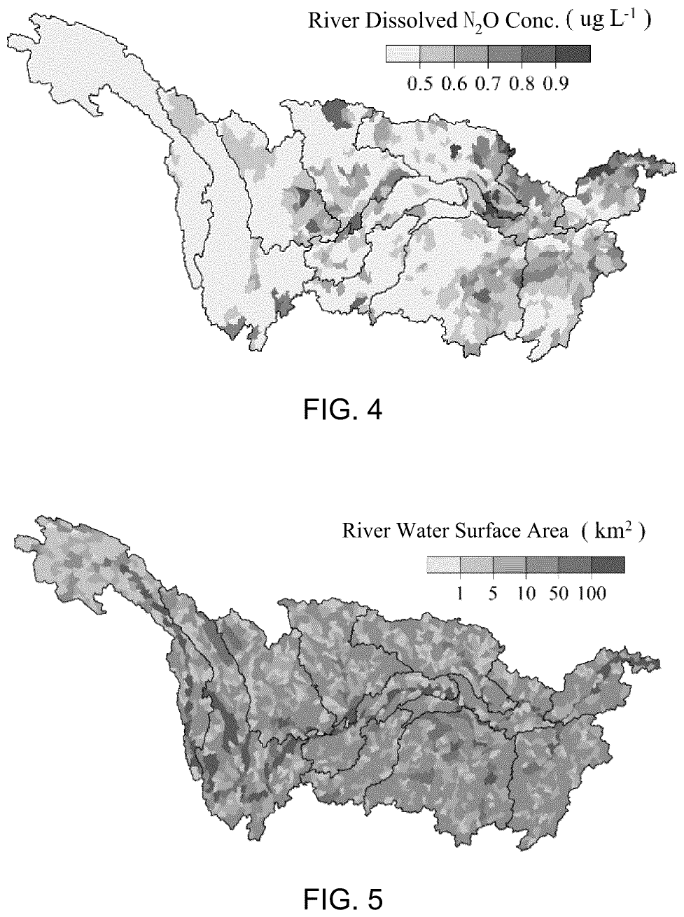

Use RF regression to predict water quality concentrations in surface water in each sub-basin of the watershed. Long-term monthly mean water quality concentrations are used as dependent variables for each sub-basin, and driver variables (geography, climate, and human activities) are used as independent variables. The training process and accuracy verification process of the model use a randomly and proportionally selected 80% and remaining 20% of the dataset. The average concentration of DIN in rivers in the Yangtze River Basin in 2019 is 1.31 mg L⁻¹. The predicted riverine DIN and dissolved N₂O concentration (processed by computer to generate outputs as images on a display) are shown in FIG. 3 and FIG. 4.

### 3. Simulation of River Hydrological State

Collect digital elevation models (DEMs) with a resolution of 90 m, a soil type map and a slope map of the Yangtze River Basin. With a threshold watershed area of 500 km², divide the watershed into sub-basins. The further divide these sub-basins into hydrological response units (HRUs) based on land use and slope. Using long-term daily climate data to drive the SWAT simulations, model preheaters are used to mitigate initial conditions during the first 3 years and are excluded from the analysis.

Using the model output values in 2019, Calculate the length, water depth, and flow velocity of the river in each sub-basin, and then calculate the water surface area of the river based on the length and average width of the river in each sub-basin. The calculation results (process by computer and output in the form of an image on a display) of the river water surface area are shown in FIG. 5.

### 4. Calculation of River N₂O Gas Emissions

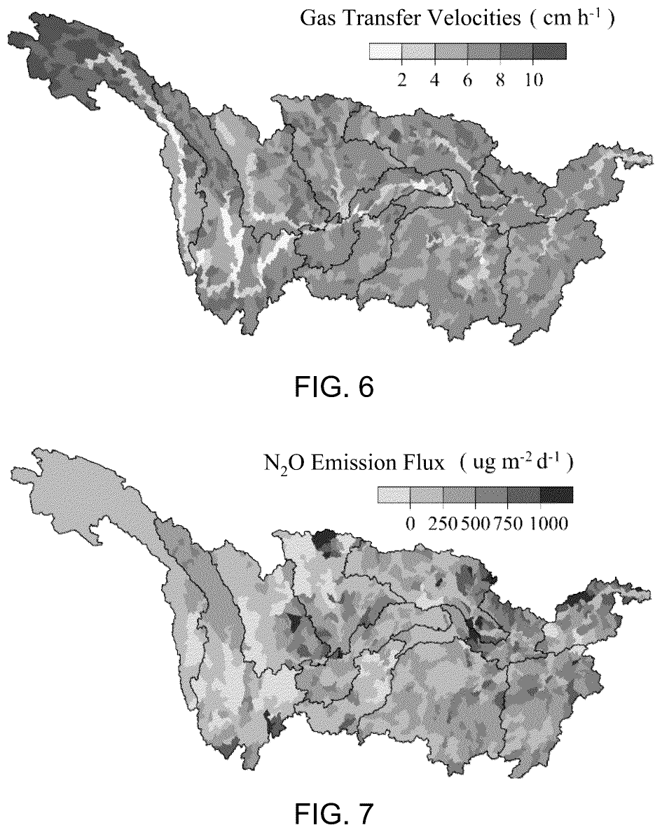

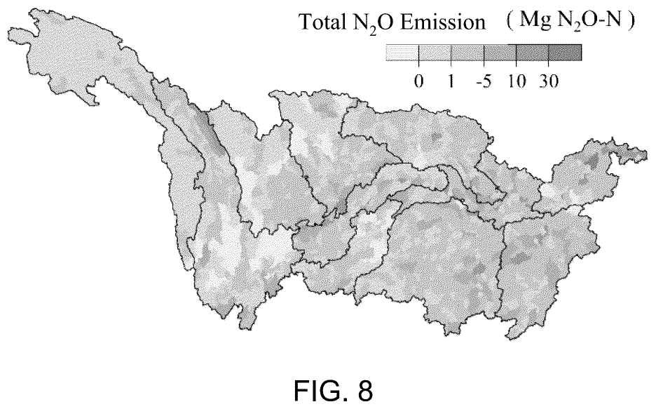

Based on the simulated value of the riverine dissolved N₂O concentration, the monitoring values of air temperature and wind speed, combined with the river water temperature output by the SWAT model, and based on the solubility equation and the air-water interface gas exchange model, calculate the gas transfer velocity and emission flux of N₂O. Together with the river water surface area simulated by the SWAT model, calculate the river N₂O emissions in the Yangtze River Basin. The total river N₂O emission in the Yangtze River Basin in 2019 is 2.8 Gg N₂O -N yr⁻¹. The gas transfer velocity simulation results (process by computer and output in the form of an image on a display) are shown in FIG. 6, and the N₂O emission flux and total emission calculation results (process by computer and output in the form of images on a display) are shown in FIGS. 7 and 8.

Although the present invention is described herein with reference to specific embodiments, it is to be understood that these embodiments are merely exemplary of the principles and applications of the present invention. It is therefore to be understood that many modifications may be made to the exemplary embodiments and other arrangements may be devised without departing from the spirit and scope of the invention as defined by the appended claims. It is to be understood that the features described in the different dependent claims may be combined in a different manner than that described in the original claims. It is also understood that features described in connection with a single embodiment can be used in other described embodiments.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of determining nitrous oxide emissions of a river based on land-river-atmosphere simulation, comprising the steps of:
(1) obtaining nitrogen emission from land in each region;
(2) dividing the nitrogen emission from land into a nitrogen emission prediction set and a nitrogen emission test set; dividing geographical variables in each region into geographical variable prediction set and geographical variable test set; dividing climate variables in each region into climate variable prediction set and climate variable test set;
training a RF regression model by using nitrogen emission prediction set, geographical variable prediction set and climate variable prediction set to obtain a trained RF regression model, inputting the nitrogen emission test set, the geographical variable test set and the climate variable test set into the trained RF regression model, and outputting a river water quality concentration of each sub-basin in each region;
(3) obtaining river hydrological parameters of each sub-basin in each region, wherein the river hydrological parameters of each sub-basin comprises a water depth of the river, a flow velocity of the river, a water temperature of the river and a water surface area of the river;
(4) providing an air-water interface gas exchange model and inputting the hydrological parameters of each sub-basin and river water quality concentration of each sub-basin in each region; and processing concentration conversion to obtain a total river N₂O emission in each sub-basin in each region.

2. The method of determining nitrous oxide emissions of a river based on land-river-atmosphere simulation according to claim 1, wherein nitrogen emissions on land comprises urban residential anthropogenic nitrogen emission, industrial anthropogenic nitrogen emission, urban stormwater runoff non-point source nitrogen emission, rural residential nitrogen emission, crop farming nitrogen emission, and livestock farming nitrogen emission.

3. The method of determining nitrous oxide emissions of a river based on land-river-atmosphere simulation according to claim 2, wherein the urban residential anthropogenic nitrogen emission refers to:
$$\{ \begin{matrix}{URN}_{discharge} = {URN}_{direct} + {URN}_{treat} \\ {URN}_{direct} = URPop \times {URCof}_{water} \times {URRate}_{direct} \times {URConc}_{direct} \\ {URN}_{treat} = URPop \times {URCof}_{water} \times {URRate}_{treat} \times ( 1 - {URRate}_{reuse} ) \times {URConc}_{treat}\end{matrix} ,$$
where URN_{discharge} refers to nitrogen discharge to water environment from urban residential area; URN_{direct} refers to a quantity of direct sewage discharge to the water environment without sewage treatment; URNₜᵣₑₐₜ refers to a quantity of nitrogen emission from urban residential area discharged to the water environment after sewage treatment by urban sewage treatment plant; URPop refers to an urban population; URCof_{water} refers to a domestic water coefficient of residents in urban area (per capita); URRate_{direct} refers to a ratio of direct sewage discharge to a total amount of sewage; URConc_{direct} refers to a nitrogen emission concentration of direct sewage discharge; URRateₜᵣₑₐₜ refers to a ratio of sewage treated by sewage treatment plants to a total sewage; URRateᵣₑᵤₛₑ refers to an effluent reuse rate of sewage treatment plant; URConcₜᵣₑₐₜ refers to a pollutant discharge concentration from the sewage treatment plant;
the urban stormwater runoff non-point source nitrogen emission refers to:
$$\{ \begin{matrix}{USRN}_{discharge} = {\sum }_{i = 1}^{4} {USRNRate}_{i} \times {UArea}_{i} \\ {USRNRate}_{i} = {NCon}_{i} \times {PDen}_{i , j} \times SF \times {AP}_{i} \times NCoef \\ {PDen}_{i , j} = 0.142 + 0.111 \times {DP}_{i}^{0.54} ( i = 1 ) \\ {PDen}_{i , j} = 1 ( i = 2 , 3 ) \\ {PDen}_{i , j} = 0.142 ( i = 4 ) \\ {UArea}_{i} = {UArea}_{2018} \times \frac{{UACoef}_{j}}{{UACoef}_{2018}}\end{matrix} ,$$
wherein USRN_{discharge} refers to a nitrogen discharge from urban stormwater runoff surface source, USRNRateᵢ refers to a runoff nitrogen emission per unit area corresponding to a functional area i, where i=1, 2, 3, 4, which correspond to living area, commercial area, industrial area and other area respectively; UAreaᵢ refers to a surface area of the functional area i; NConᵢ refers to a nitrogen emission concentration of the functional area i; PDen_{i, j} refers to an urban population density parameter of the functional area i in year j; SF refers to a cleaning frequency of urban community, where cleaning once a day is defined as 1; APᵢ refers to an annual precipitation (cm) in year j of the city where the functional area is located; NCoef refers to a nitrogen emission correction coefficient; PDen_{i, j} is a correction coefficient of the functional area; DP^{0.54}ᵢ is a population density of an administrative region; UACoefⱼ refers to a correction coefficient of urban surface area in year j, UACoef₂₀₁₈ refers to a correction coefficient for urban surface area in 2018, UArea₂₀₁₈ refers to a surface area of the functional area in 2018;
the rural residential nitrogen emission refers to:
$$\{ \begin{matrix}{RRN}_{discharge} = {RRN}_{direct} + {RRN}_{treat} \\ {RRN}_{direct} = {RRN}_{total} \times {RRRate}_{direct} \\ {RRN}_{treat} = {RRN}_{total} \times {RRRate}_{treat} \times ( 1 - {RRCoef}_{removal} ) \\ {RRN}_{total} = RRPop \times ( {RRRate}_{DryT} \times {RRCoef}_{DryT} + {RRRate}_{FlushT} \times {RRCoef}_{FlushT} )\end{matrix} ,$$
where RRN_{discharge} refers to a nitrogen discharge to the water environment from rural residential; RRN_{direct} refers to a quantity of direct sewage discharge to the water environment without sewage treatment; RRNₜᵣₑₐₜ refers to a pollution discharge released to the water environment after treatment by rural sewage treatment facility; RRRateₜᵣₑₐₜ refers to a ratio of sewage treated by rural sewage treatment facility to a total sewage in rural area; RRCoefᵣₑₘₒᵥₐₗ refers to a pollutant removal rate of rural sewage treatment; RRPop refers to a total number of residents in rural area; RRRate_{DryT} refers to a ratio of dry toilets to total toilets in rural area; RRCoef_{DryT} refers to per capita pollutant emission coefficient of residents in rural area who use dry toilet; RRRate_{FlushT} refers to a ratio of flush toilets to total toilets in rural area; RRCoef_{FlushT} refers to a pollutant emission coefficient of residents in rural area who use flush toilet;
the crop farming nitrogen emission refers to:
$$\{ \begin{matrix}{CFN}_{discharge} = CFArea \times {CFCoef}_{discharge} \\ {CFCoef}_{discharge} = {CFCoef}_{discharge , 2017} \times \frac{{CFFertilizer}_{i}}{{CFFertilizer}_{2017}}\end{matrix} ,$$
where CFN_{discharge} refers to a pollution emission discharged into the water environment from crop farming; CFArea refers to a total sown area of farmland; CFCoef_{discharge} is a pollutant loss coefficient of farmland; CFCoef_{discharge,2017} refers to a standard nitrogen loss coefficient of farmland based on data of the China's Second Pollution Source Census in 2017; CFFertilizerᵢ refers to a quantity of chemical fertilizer application in year i; CFFertilizer₂₀₁₇ refers to a quantity of chemical fertilizer application in 2017;
the livestock farming nitrogen emission refers to:
$$\{ \begin{matrix}{LFN}_{discharge} = {LFN}_{centralized} + {LFN}_{free} \\ {LFN}_{centralized} = {\sum }_{i = 1}^{6} {LFNumber}_{i} \times {LFRate}_{centralized , i} \times {LFCoef}_{centralized , i} \\ {LFN}_{free} = {\sum }_{i = 1}^{6} {LFNumber}_{i} \times {LFRate}_{free , i} \times {LFCoef}_{free , i}\end{matrix} ,$$
where LFN_{discharge} refers to a nitrogen discharge to the water environment from livestock farming; LFN_{centralized} is a pollution emission from centralized livestock farming; LFN_{free} is a pollution emission from free-range livestock farming; LFNumberᵢ is the number of fattening livestock i; LFRate_{centralized, i} is a ratio of centralized breeding quantity to total quantity of species i; LFCoef_{centralized, i} is a nitrogen emission coefficient of species i in centralized farming; LFRate_{free, i} is a ratio of free-range quantity to total quantity of species i; LFCoef_{free, i} is a nitrogen emission coefficient of species i in free-range livestock;
wherein the industrial anthropogenic nitrogen emission is obtained by: obtaining industrial nitrogen emissions in each region, and according to the proportion of the area of each sub-basin in each region to the region, converting the industrial nitrogen emissions in each region into the nitrogen emissions of each sub-basin, which is defined as the nitrogen discharged by industry into the water environment.

4. The method of determining nitrous oxide emissions of a river based on land-river-atmosphere simulation according to claim 1, wherein in step (2), the step of outputting a water quality concentration of each sub-basin in each region of the river comprises the sub-steps of:
dividing the nitrogen emissions from the land in each region into nitrogen emission prediction set and nitrogen emission test set, dividing the environmental investment data in each region into an environmental investment data prediction set and an environmental investment data test set, and dividing the social statistical data on population and economy in each region into a prediction set of the social statistical data on population and economy, and a test set of the social statistical data on population and economy;
training the RF regression model by using the nitrogen emission prediction set, geographical variable prediction set, climate variable prediction set, environmental investment data prediction set, the prediction set of the social statistical data of population and economy to obtain the trained RF regression model; and then inputting the nitrogen emission test set, geographical variable test set, climate variable test set, environmental investment data test set, the test set of the social statistical data of population and economy to obtain the trained RF regression model and output the river water quality concentration of each sub-basin in each region.

5. The method of determining nitrous oxide emissions of a river based on land-river-atmosphere simulation according to claim 4, wherein the environmental investment data comprises a proportion of environmental pollution investment and a number of environmental regulations;
the social statistical data on population and economy comprises a population density, a gross national product, a quantity of fertilizer application, a number of mobile phone households and a length of graded highway kilometers;
the geographic variables comprise a soil bulk density, soil organic matter, soil conductivity, soil pH, soil type proportion, land use proportion, maximum patch index, edge density, landscape shape index, Shannon diversity index and median landscape perimeter-to-area ratio;
the climate variables comprise an average temperature, an accumulated temperature greater than 10° C., an average rainfall, a humidity index and a normalized vegetation index.

6. The method of determining nitrous oxide emissions of a river based on land-river-atmosphere simulation according to claim 1, wherein in step (3), the step of obtaining hydrological parameters of each sub-basin in each region of the river comprises the sub-steps of:
inputting climate data of each sub-basin in each region into a SWAT model for hydrological parameter simulation, then outputting the river hydrological parameters of each sub-basin, wherein the climate data comprises rainfall temperature data, wind speed data, relative humidity data and solar radiation data.

7. The method of determining nitrous oxide emissions of a river based on land-river-atmosphere simulation according to claim 1, wherein the total river N₂O emission in each sub-basin refers to:
$$\{ \begin{matrix}{F}_{{N}_{2} O} = {k}_{{N}_{2} O} \times ( {C}_{w} - {C}_{eq} ) \times 240 \\ {F}_{{N}_{2} Ototal} = {\sum }_{sub - basin} ( {\sum }_{month} {F}_{{N}_{2} O} \times SA \times N \times {10}^{- 15} ) \\ {C}_{w} = 0.42 \times {DIN}^{0.61} \\ {C}_{eq} = {C}_{air} \times {e}^{[ - 165.8806 + 222.8743 \times \frac{100}{{T}_{K}} + 92.0792 \times l n \frac{{T}_{K}}{100} - 1.48425 \times {( \frac{{T}_{K}}{100} )}^{2} ]} \times {M}_{{N}_{2} O} \\ {k}_{{N}_{2} O} = {k}_{600} \times {( \frac{{Sc}_{{N}_{2} O}}{600} )}^{- n} \\ {Sc}_{{N}_{2} O} = 2055.6 - 137.11 \times T + 4.3173 \times {T}^{2} - 0.05435 \times {T}^{3}\end{matrix} ,$$
where F_{N}_{₂}_{O} is a N₂O emission flux from the river to the atmosphere; C_{w} is the dissolved N₂O concentration in surface water of the river; C_{eq} is a theoretical N₂O concentration in surface water and atmospheric N₂O balance; k_{N}_{₂}_{O} is a N₂O gas transfer velocity; 240 is a unit conversion factor; F_{N}_{₂}_{Ototal} is a total N₂O emission in the basin in a given year; SA is a water surface area in a given sub-basin; N is a number of days in a given month; DIN is a dissolved inorganic nitrogen concentration simulated by the RF regression model; T_{K} is a water temperature simulated by SWAT; M_{N}_{₂}_{O} is a molecular weight of N₂O in N; Cₐᵢᵣ is a monthly scale N₂O concentration in air calculated based on a N₂O combined data set provided by NOAA Global Monitoring Laboratory; Sc_{N}_{₂}_{O} is a Schmidt number, T is a water temperature; n is an index; k₆₀₀ is a gas transfer velocity at 20° C.

8. The method of determining nitrous oxide emissions of a river based on land-river-atmosphere simulation according to claim 7, wherein n is ⅔, k₆₀₀=2.07+0.215×W₁₀^{1.7}, W₁₀ is a wind speed at 10 m height.

9. The method of determining nitrous oxide emissions of a river based on land-river-atmosphere simulation according to claim 7, wherein n is ½, k₆₀₀=1.0+1.719×(V/H)^{0.5})+2.58×W₁₀, W₁₀ is a wind speed at 10 m height, V and H are flow velocity and water depth respectively.
